(19) United States
(12) Patent Application Publication
(10) Pub. No.: US 2007/0135886 A1
(43) Pub. Date: Jun. 14, 2007

(54) Catheter device

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/634,035

(22) Filed: Dec. 5, 2006

(30) Foreign Application Priority Data
Dec. 12, 2005 (DE) 10 2005 059 271.6

(51) Int. Cl. A61F 2/06

(52) U.S. Cl. 623/1.11

(57) ABSTRACT

The invention relates to a catheter device for performing an atherectomy, which device contains an atherectomy catheter and a stent premounted on the atherectomy catheter.

### CROSS REFERENCE TO RELATED APPLICATIONS

This application claims priority of German application No. 10 2005 059 271.6 filed Dec. 12, 2005, which is incorporated by reference herein in its entirety.

### FIELD OF THE INVENTION

The invention relates to a catheter device for performing an atherectomy.

### BACKGROUND OF THE INVENTION

Vascular diseases are among the commonest disorders having a fatal outcome. Featuring most prominently is myocardial infarction due to diseased coronary vessels. When coronary vessels have become blocked by arteriosclerotic plaque, this clinical condition is customarily treated using percutaneous transluminal coronary angioplasty (PCTA) whereby the narrowed parts of the coronary vessels are dilated using a balloon catheter. However, clinical studies have shown that restenosing occurs in very many patients, with in some cases 50% of patients exhibiting this. An alternative method that has been known for a number of years is high-frequency rotablation angioplasty, which can be advantageously applied especially to fibrotic or sclerotic or long-segment stenoses.

To reduce the risk of restenosing, coronary atherectomy is employed to recanalize stenosed coronary arteries through what is termed debulking. The device used for performing the atherectomy is a catheter system having a metal housing accommodating the actual excising apparatus, what is termed the cutter. The cutter, consisting of a conically ground knife, is linked to a motor external to the patient via a flexible connection. The knife is driven by said motor to rotate at a speed of 1,500 to 2,000 rpm. Mounted on one side of the metal housing is a balloon; on the contralateral side is a window. The balloon is inflated during the atherectomy, thereby pressing the openings and knife into the plaque. The rotating knife can then be externally pushed forward toward the tip of the atherectomy housing. As a result, the plaque will be excised and the plaque material pushed to the tip of the atherectomy housing. The balloon is then deflated, the atherectomy device rotated a little so that the window points in another direction of the plaque, and the process repeated. An atherectomy device is known from U.S. Pat. No. 5,895,402.

To keep the vessel open, it is often necessary in the treatment of vascular diseases to insert a stent, which is a vessel support that mechanically stabilizes the vessel wall. The use of stents allows the vessel to be further dilated, for example. To insert such stents it is necessary first to remove the catheter on which the atherectomy instrument for treating the vasoconstriction is provided then insert the stent using a second catheter. That, though, is a procedure that puts a strain on the patient and entails risks, particularly in terms of restenosing.

What has been proposed in US 2005/0203553 A1 is a catheter having an integrated OCT sensor for use in blood vessels and by means of which sensor the quality of imaging in the vicinity of the stenosis will be improved.

What has been proposed by US 2005/0101859 A1 is a medical investigation and/or treatment system that combines the OCT and IVUS imaging methods in a single device. That allows overlaid 2D image recordings to be produced, with the OCT image section being used for the near area and the IVUS image section being used for the far area.

A medical investigation and/or treatment system is known from US 2005/0113685 A1 wherein the OCT and IVUS imaging methods are combined in a catheter additionally provided with a position sensor. Three-dimensional recordings can be produced using the information registered by said position sensor.

What is common to all known solutions is that they each only resolve single issues. Hitherto it has not been possible to optimally integrate the conventional catheters into the medical workflow.

### SUMMARY OF THE INVENTION

The problem underlying the invention is thus to provide a catheter device that is better integrated into the medical workflow and by means of which it can be made easier to insert a stent.

To resolve said problem it is inventively provided for a catheter device of the type mentioned in the introduction to include an atherectomy catheter and a premounted stent on the atherectomy catheter.

The catheter device can furthermore have an OCT sensor, an IVUS sensor, or, as the case may be, position sensors, as well as an image processing unit embodied, where applicable, for producing combined 2D and/or 3D image recordings on the basis of the sensors' data.

That will allow optimal diagnostic imaging as part of minimally invasive medical therapy.

The invention is based on the knowledge that it has hitherto only been possible to combine separately used catheters into an integrated structural unit by employing an atherectomy catheter and a premounted stent as well as, where applicable, an IVUS sensor, an OCT sensor, and position sensors, and overlaying the image information obtained therefrom in a 2D representation or, as the case may be, using said information to produce a 3D image recording.

The combination catheter has a premounted stent serving to support the vessel. Because such a stent—also understood, of course, as meaning a plurality of separate devices or, as the case may be, stents serving as a vessel support—is likewise arranged on the single integrated catheter device, there is no need to afterwards remove a catheter that has been used for, for instance, clearing the plaque as part of an atherectomy. The stent can be inserted by means of the integrated catheter device simultaneously with the therapy implements. A significantly reduced risk of restenosing ensues therefrom.

In all, therefore, therapy is made possible using a single catheter with which both the vascular occlusion can be removed, where applicable accompanied by appropriate image monitoring, and a stent maintaining the vessel's open condition can be inserted therein. The therapy will thus require fewer procedural steps, with there also being the possibility, where applicable, of monitoring the process by means of three-dimensional image recordings. Representation of the near area will be insured when IVUS, OCT, and position sensors have been combined, while adequate images of deeper tissue layers will be obtained at the same time. Utilizing the signals of the position sensor system will allow the location and motion of the integrated catheter for the atherectomy therapy to be imaged with the aid of the IVUS signals, OCT signals, and the cited—for instance electromagnetic—signals of the position sensor system so that the x-radiation to which the patient is exposed can be reduced.

The stent can be premounted in the vicinity of the catheter's tip. The device for supporting the vessel will thus from the outset be located in the area undergoing therapy so that the stent can then, without moving the catheter significantly, be positioned at the right place where the atherectomy therapy was performed.

An expansible balloon can furthermore be provided in the vicinity of the catheter's tip, with the premounted stent being able to be positioned and/or secured in position as a function of said balloon's expansion. The stent will thus be pressed into the vessel wall, for example, and thereby secured in position when the balloon is filled with compressed air. The stent can in its undeployed condition be located on the balloon or, as the case may be, in the vicinity thereof so that the stent's location relative to the vessel will be influenced when the balloon is expanded. For example, when the balloon is inflated the stent can be deformed beyond its elastic limits or, as the case may be, overstretched so that the shape resulting from the balloon's being inflated will afterwards be retained. With the aid of the balloon, the stent will consequently be selectively deformed and positioned or, as the case may be, secured in position or anchored in the area of the vessel.

The stent can also be embodied as being at least partially self-deploying. In this case a cladding, for example, made of a plastic material and at least partially surrounding the stent will be removed, whereupon the relevant area of the stent will open out. As a rule, either a stent opening out with the aid of a balloon or a self-deploying stent is used. It is, though, also conceivable for both these possibilities for inserting the stent or, as the case may be, securing it in position in the area of the vessel to be combined.

The stent can, moreover, be embodied at least partially from metal, in particular high-grade steel or nitinol. Lattice-type or mesh-type arrangements consisting of, for example, steel or a specific metal or other metal alloys, for example the nickel-titanium alloy nitinol or another shape-memory alloy, are as a rule used for stents.

The stent can also be embodied at least partially from bioresorbable material, in particular biologic material and/or magnesium and/or bio-engineering material and/or plastic. For example polymers can be used. Bioresorbable materials have the advantage of disintegrating after a certain, possibly predefined period of time so that, when no longer necessary as a vessel support after a certain period of time, the stent will degrade automatically and so be removed, with no further intervention and posing no risk for the patient. Other advantageous materials and combinations of materials can, of course, also be used for the stent that impact positively on the vessel's inner surface or, as the case may be, can support the vessel and maintain its open condition. Furthermore, requirements have to be adhered to regarding the possibilities for insertion as well as for visualizing for medical check-ups, for instance. Alongside this, the stent materials' properties have to be taken into account in terms of their effect on blood flow or blood clotting.

The stent is advantageously embodied as being coated, in particular with a nano coating and/or active component coating. Coatings of said type make it possible to improve, for example, guiding of the catheter device on which the stent or stents is/are premounted. A coating containing active components or medicaments that will be released over a certain period of time or at a specific time is used, for example, in order to control the division of the vessel wall's cells. Moreover, the risk of restenosing can be further reduced by way of appropriate active components or medicaments that are released once the stent has been positioned in the area of the vessel.

The active component coating can contain Sirolimus and/or Paclitaxel and/or Everolimus and/or Rapamycin and/or FK 506 or, where applicable, a combination thereof. Other growth inhibitors are likewise suitable.

The catheter can furthermore be embodied having an automatic advancing and/or withdrawing device. That will enable the integrated atherectomy catheter to be inserted into or, as the case may be, withdrawn from the vessels at a defined speed, as a result of which for example complications due to overhasty or imprecise manual guiding can be avoided.

It is preferable for the inventive catheter device to be integrated in a medical therapy device, in particular an x-ray device. An angiographic or cardiological x-ray system of said type having a high-voltage generator, an x-ray machine, a radiation diaphragm, an image detector unit, an operating table, radiation source and detector stands, and a digital imaging system will make it possible to produce angiographic radiograms as well as image recordings of the nature of computer-assisted tomograms, and will be capable of processing, representing, and overlaying the information and image recordings supplied by the inventive catheter device.

A magnetic control, but alternatively also a mechanical control, preferably having pull wires for displacing the tip of the catheter can be provided for the inventive catheter device. The tip of the catheter can in this way be displaced to one side.

It can also be provided for the catheter to be controllable by means of an external magnetic field, with the catheter having at least one permanent magnet and/or at least one electromagnet. Receiver coils can in a further embodiment of the invention have iron cores and optionally be employed as a receiving antenna or an electromagnet for magnetic navigation.

To achieve miniaturizing of the catheter it is not necessary for the coils to be arranged mutually orthogonally; rather they can also be arranged at any angle, in particular an angle of about 60°.

The OCT sensor and/or IVUS sensor can in the inventive catheter device be oriented to the side, referred to the catheter's longitudinal axis. The OCT sensor and IVUS sensor can accordingly be rotatable separately or jointly around the catheter's longitudinal axis. It is, though, alternatively also possible to provide a plurality of sensors that are disposed in fixed, circumferential positions and interrogated sequentially. It is also possible for the catheter to be capable of being advanced or withdrawn at a definable speed by means of a drive unit. Three-dimensional image recordings can be produced in this way.

The image processing unit of the inventive catheter device can within the scope of image processing be embodied for approximating the center line and/or envelope curve of a part of the body, in particular a vessel, being examined. The vessel's envelope curve can be used in further image post-processing steps. For example, the three-dimensional OCT-IVUS image recordings can with the aid of the envelope curve be registered along with other anatomic image data originating, say, from a 3D angiography system, then displayed in merged form, with the 3D image recordings of the catheter and the anatomic image data being expediently translated into a common system of coordinates.

To keep the inventive catheter device free from motion artifacts caused by, for example, breathing or the motion of the heart or other organs, the frequency and/or amplitude of the motion can be registered and computationally corrected.

To avoid interference when the sensors are registering signals, it can be provided for said sensors to be capable of being read out in time-lagged clocked fashion. For example, x-ray detectors and a possibly present electrocardiogram will not be read out when the transmitters of the electromagnetic positioning system are active; the OCT sensors and position sensors will not be read out when x-radiation is active. So only signals that will not be affected by interference will be registered in each case.

Particularly good results can be achieved when the inventive catheter device has a coating for screening electromagnetic fields. A coating of said type can have a thin-film layer consisting of conductive nano particles.

The catheter and its sensors can be electrically decoupled from the power line voltage so that this will pose no risk to the patient.

The catheter can have x-ray markers to make the catheter easier to locate using radiograms.

The catheter can be provided with a coating consisting preferably of a silicon material and/or nano materials to reduce its frictional resistance while it is being moved inside a vessel. The catheter can have an inflatable balloon, particularly at its tip, to support positioning.

In order to be able to issue a warning in the event of, where applicable, increases in temperature, the catheter can have a temperature sensor or, where applicable, also a pressure sensor arranged preferably at its tip.

The invention relates also to a medical therapy device, in particular an x-ray device. The inventive therapy device includes a catheter device of the type described.

The invention relates also to a method for producing investigation images while an atherectomy is being performed. The inventive method is characterized in that an atherectomy catheter is used having an OCT sensor, an IVUS sensor, and position sensors, as well as a premounted stent, with combined 2D and/or 3D image recordings based on the sensors' data being produced by means of an image processing unit.

### BRIEF DESCRIPTION OF THE DRAWINGS

Further advantages and specifics of the invention will be explained with the aid of exemplary embodiments and with reference to the figures. The figures are schematic representations and show:

FIG. 1 an inventive catheter device for performing an atherectomy;

FIG. 2 and 3 a second exemplary embodiment of an inventive catheter device;

FIG. 4 an inventive therapy device having a catheter device; and

FIG. 5 a schematic of the sensor readout produced using the therapy device shown in FIG. 4.

### DETAILED DESCRIPTION OF THE INVENTION

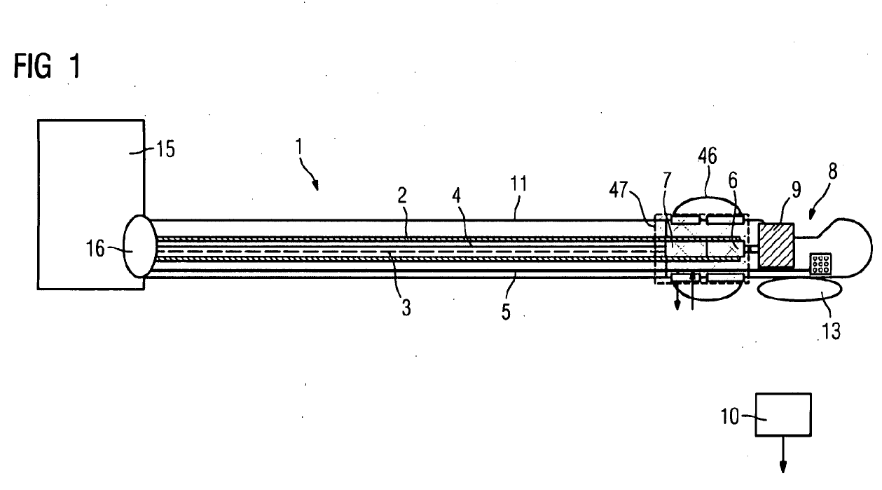

FIG. 1 shows an inventive catheter device 1 embodied as an atherectomy catheter. The inventive catheter device 1 has a hollow flexible drive shaft 2 in which an OCT signal lead 3 and an IVUS signal lead 4 are integrated. A signal lead 5 for a position sensor system embodied as an electromagnetic sensor system is furthermore arranged in the flexible drive shaft 2. An IVUS sensor 6 and an OCT sensor 7 are integrated in the front part of the catheter. An opening having a cutter 9 embodied as a rotating knife is located in the vicinity of the tip 8 of the catheter. A light-exit window for the OCT sensor 7 is located at the tip 8 of the catheter. Magnetic sensors of the sensor system are also located there. Said sensors interact with a position sensor 10 located outside the patient's body. The position sensor 10 is embodied as an electromagnetic sensor.

The catheter device moreover has a premounted stent 47 that is embodied as a metal-wire mesh and has here been sketched in its non-expanded position. When the cutter 9 has been deployed for clearing the plaque, the stent 47 will be expanded by means of the expansible balloon 46, whose feeders have not been shown here in the interest of greater clarity. The balloon 46 is filled so that the stent 47 arranged thereon will be expanded in the direction of the vessel wall and pressed into it. The positioning of the stent 47 is monitored with the aid of the imaging sensors.

The inventive catheter device 1 thus allows an atherectomy to be performed on a vascular occlusion accompanied by optimal image monitoring by OCT and IVUS in combination with position sensors, and a stent to be positioned in the vessel during this procedure without having to insert separate catheters. When the vascular occlusion has been cleared with the aid of the cutter 9, the premounted stent 47 will be opened out as a function of the filling of the balloon 46 and arranged in the vessel to support it. The stent 47 is provided with a medicament coating via which a defined amount of a medicament is released to prevent restenosing. The structure of the stent 47 being known, imaging during the atherectomy will not be adversely affected thereby.

The drive shaft 2 is surrounded by a catheter mantle 11. Opposite the opening is an expansible balloon 13 for supporting positioning.

A signal interface and a drive unit 15 are connected to the catheter device 1 via a rotary coupling 16.

In the case of the catheter device 1 shown in FIG. 1 the cutter 9 for performing an atherectomy is linked to the OCT sensor 7, the IVUS sensor 6, and position sensors to produce an integrated device.

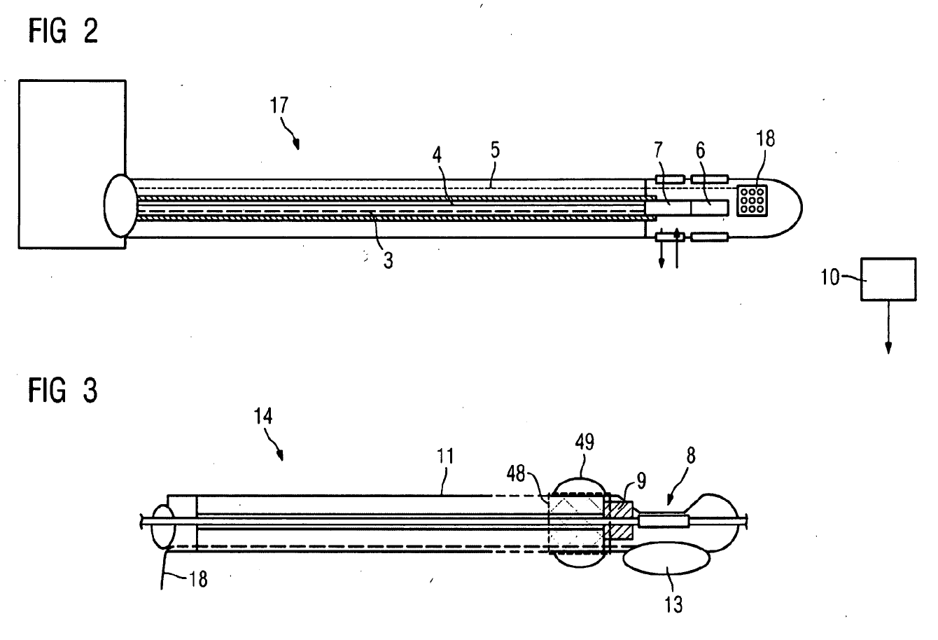

FIGS. 2 and 3 show a second exemplary embodiment of a catheter device.

The same reference numerals are used for components of the catheter device that tally with those of the first exemplary embodiment.

FIG. 2 shows an imaging catheter 17 having an IVUS sensor 6, an OCT sensor 7 with an inspection window, position sensors, signal leads 4 for IVUS, and signal leads 3 for OCT. Also provided are a signal interface and a drive unit 15.

FIG. 3 shows an atherectomy catheter 14 having a lumen into which the imaging catheter 17 can be inserted. Like the catheter shown in FIG. 1, the atherectomy catheter 14 has a cutter 9 in the vicinity of the tip 8 of the catheter and an expansible balloon 13. The lumen is transparent for OCT and IVUS in the vicinity of the tip 8 of the catheter. Inside the catheter 14 is a tube 18 for a pressurizing agent for the balloon 13.

The atherectomy catheter 14 furthermore has a premounted stent 48 that is opened out by means of an expansible balloon 49, which is a high-pressure balloon, as a function of said balloon's expansion. The premounted stent 48, whose expansion is not shown here, is provided with a nano coating in order not to adversely affect the guiding of the atherectomy catheter 14. It is thus inventively possible to completely treat the vascular occlusion, including applying the stent, solely by means of the atherectomy catheter 14 having the inserted imaging catheter 17, with no withdrawing necessary or, as the case may be, no need to re-insert further catheters.

The two catheter devices 1, 17 shown in FIGS. 1 to 3 each have an OCT sensor and an IVUS sensor. The OCT sensor supplies particularly good images of the near area; the IVUS sensor provides a good representation of more distant or, as the case may be, deeper layers.

The catheter devices 1, 17 are connected to an image processing unit which produces a joint image from the images supplied by both sensors. For this purpose a section of the image supplied by the OCT sensor is used for the near area and the complementary part of the IVUS image is used for the far area, then the two sections are mutually registered by means of the position sensors' data and merged into a joint image. In this way, cross-sectional images precisely assignable to a specific location in the body are obtained of the vessel being examined. Through the application of computational methods the position sensor's data is used to approximate the center line and envelope curve of the vessel being examined. The individual cross-sectional images are then combined into a volume dataset so as to yield an exact and hence especially realistic image.

The geometric information of the center line is used in approximating the vessel's center line and envelope curve and combined with the sensor positions registered during image recording, as a result of which the artifacts will be significantly reduced in the 3D image presentation. The center line's 3D coordinates and the sensor positions registered during image recording are subtracted from each other. The subtraction result will then be used for each of the registered 2D images for an exact 3D reconstruction. Said envelope curve of the vessel can be used for further image processing steps. The 3D reconstructed OCT-IVUS images are registered with the aid of the envelope curve with other anatomic image data from, say, a 3D angiography device, of the same vessel section and then merged.

The position sensors 10 used for the exemplary embodiments shown in FIGS. 1 to 3 are electromagnetic position sensors for producing 3D OCT-IVUS recordings from the 2D OCT-IVUS recordings. The catheter's orientation and position in a three-dimensional system of coordinates are registered by transmitting coils in the object concerned and receiver coils in the open or, vice versa, by means of receiver coils in the object concerned and transmitting coils in the open.

The electromagnetic transmitters, or alternatively the electromagnetic receivers, can be located in the catheter. Vice versa, the corresponding electromagnetic receivers or transmitters can be located outside the body. Usually at least one transmitter emitting in the X, Y, Z direction is assigned to a receiver or, vice versa, one receiver having X, Y, Z receive directions is assigned to a transmitter to enable spatial locating. The coils of the electromagnetic position sensors are not arranged exclusively mutually orthogonally but at any angle, for example an angle of 60°, in order to achieve better miniaturizing that will enable position sensors to be integrated in a catheter.

The catheter's image information recorded by means of the sensors is combined with or, as the case may be, overlaid by other medical images such as 2D or 3D recordings. The catheter's OCT-IVUS images are presented together with the radiograms. The information about the images of the catheter device and the x-ray images is thereby jointly visualized for the user, enabling faster and better diagnosing. 2D-2D, 2D-3D, 3D-3D as well as 3D4D and 4D-4D overlays are also possible, with the angiographic x-ray images being in each case combined with the catheter device's images by means of segmenting, registering, and image merging. Images obtained using the following modalities and methods can be employed for overlaying: Sonography including IVUS, radiography, fluoroscopy, angiography, OCT, discrete tomography, positron-emission tomography, nuclear medical diagnostics, computer-assisted tomography, nuclear magnetic resonance tomography including intracardial MR, optical recordings including endoscopy, fluorescence, and optical markers.

The catheter device is part of a medical therapy device having a functional unit for eliminating motion artifacts caused by breathing or motion of the heart or blood vessels. To eliminate breathing artifacts, it is also possible to use a chest band that determines the amplitude and frequency of breathing via suitable sensors so that the image processing unit can calculate appropriate corrections in order to computationally eliminate motion artifacts from the image information.

To increase the accuracy of locating, the transmitting coils are operated and evaluated cyclically during specific time segments and at different frequencies. To avoid sensor artifacts that can be caused by overlaying of the individual sensors' signals it is proposed reading out the sensors in time-lagged clocked fashion. For example, the x-ray detectors and ECG will not be read out when the electromagnetic positioning system's transmitters are active; the OCT sensors and position sensors will not be read out when x-radiation is active. So only signals that will sustain no interference and will not affect any other active sensors will be read out.

The functional units and signal leads are provided with devices and measures that shield physiological and image signals as well as signal processing and signal editing devices from the transmitting antennas' magnetic fields. The catheter's cladding is for this purpose coated with a thin-film layer consisting of conductive nano particles. Nano particles can also be used to provide magnetic screening.

The catheter's cladding is provided with a coating that reduces frictional resistance while the catheter is being guided through the vessels. Said coating can likewise be based on nanotechnology or, alternatively, be made from a silicon material.

To improve the IVUS sensor's imaging through the use of an ultrasound contrast medium, a contrast medium is introduced directly into the vessel being examined or, as the case may be, into the body cavity through a channel in the catheter.

A temperature or pressure sensor is arranged in the tip of the catheter for monitoring the temperature and pressure in the vessel or organ being examined and treated. A possible increase in temperature due to friction can be registered by the temperature sensor located in the tip of the catheter.

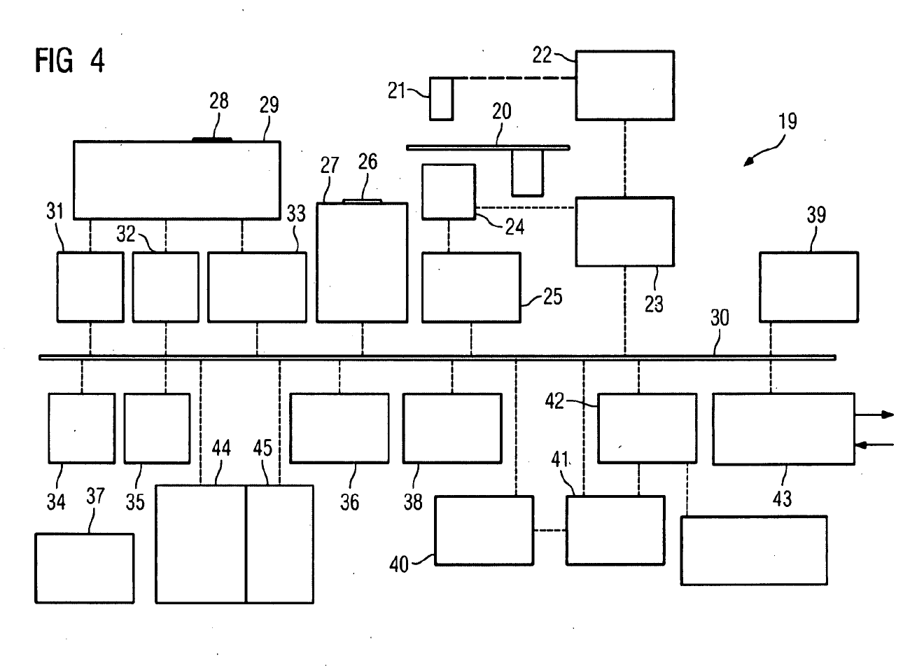

FIG. 4 is a schematic of the inventive therapy device.

The therapy device 19 includes a catheter device for performing an atherectomy. For the therapy, a patient (not shown in FIG. 4) is made to lie on an operating table 20 and radiation is emitted from a radiation source 21 in the direction of the operating table 20. The radiation is produced by means of a high-voltage generator 22 controlled via a system control 23. Arranged opposite the radiation source 21 is an x-ray detector 24, in turn connected to a preprocessing unit 25 for x-ray images. Provided in addition is a terminal 26 for physiological sensors that is coupled to a physiological signal processor 27 for controlling ECG signals or pulse signals or, as the case may be, a patient's breathing and blood pressure.

The therapy itself is performed, accompanied by image monitoring using OCT, IVUS and the electromagnetic position sensor system, via a terminal 28 for the atherectomy catheter over a signal interface 29 and is finalized by the expansion or, as the case may be, opening up of a premounted stent by means of a high-pressure balloon. There is also a connection to a data bus 30. Provided in addition are preprocessing units 31, 32, and 33 for OCT, IVUS, and the position sensors. Associated image processing units 34, 35, and 36 for OCT, IVUS, and the position sensors are likewise connected to the data bus 30. Power is supplied via a power supply unit 37. An image processing unit 38 for the x-ray images is furthermore connected to the data bus 30, which additionally has a connection to an image data memory 39 for filing and storing the recorded images. A calibration unit 40 as well as an image correcting unit 41 enable interference fields or, as the case may be, artifacts in the imaging to be taken into account. Image merging and reconstructing take place in an image merging unit and/or reconstruction unit 42. Provided in addition is an interface 43 to a patient data and image data system.

The image data obtained from OCT, IVUS, and the position sensor system as well as the x-ray images and possible merged images obtained using the various image recording techniques are presented two-dimensionally, three-dimensionally, or four-dimensionally on a display unit 44. The display unit 44 is connected to an input unit 45 for user inputs.

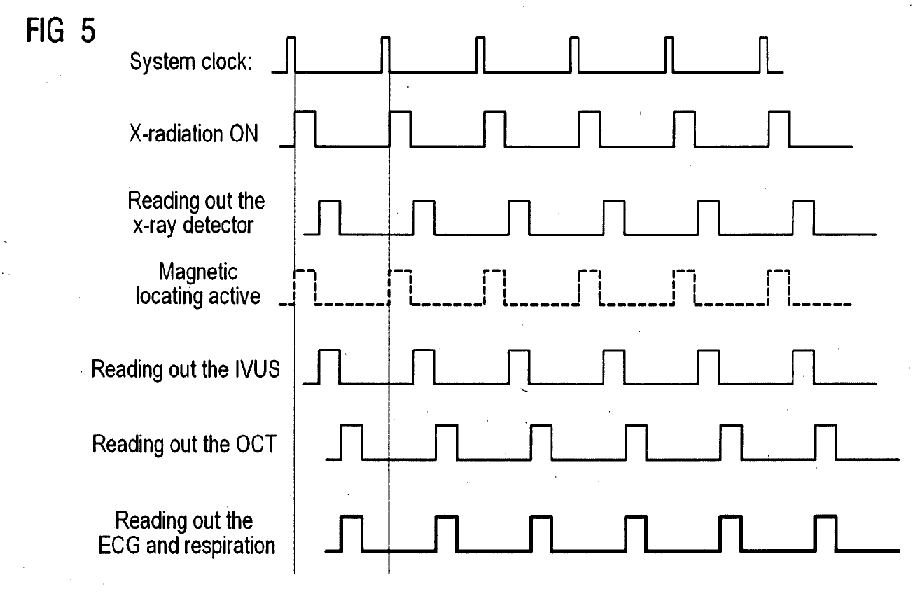

FIG. 5 is a schematic of the sensor readout produced using the therapy device when the inventive method is applied.

A typical procedural flow is as follows: Inserting the catheter under x-ray control, possibly using a contrast medium, producing the general angiographic recording, producing the recordings of the position sensors, overlaying the position sensors' recordings with the general angiograph through segmenting, registering, and image merging, and navigating the catheter, on the basis of the recordings obtained, up to the target location. These steps are performed partly in parallel and automatically with no user interaction. When the desired target location has been reached, the rinsing fluid for OCT is injected and the stenosis observed two-dimensionally or three-dimensionally at a high resolution using the OCT-IVUS image recordings. The OCT-IVUS recordings are then produced. The OCT-IVUS recordings are subsequently overlaid with the general angiograph through segmenting, registering, and image merging. The OCT-IVUS recordings are then three-dimensionally reconstructed on the basis of the position sensors' data. The atherectomy catheter is placed in position and provisionally secured by, for example, inflating the balloon attached to the tip of the catheter. Carrying out a check using OCT-IVUS in 2D and 3D to determine whether the atherectomy catheter is correctly located and positioned. Performing the atherectomy, which means scraping the plaque from the vessel wall by means of the rotating knives. The place in the vessel wall is checked using the OCT sensor when a certain amount of plaque has been removed. This process is repeated until the plaque has been removed at every place. Carrying out a final check on the atherectomy, positioning and opening out the stent until it has been secured in the vessel wall, and withdrawing the catheter.

The required procedural steps are reduced in number thanks to the inventive device. The OCT sensor supplies good recordings in the near area; the IVUS sensor supplies adequate images of deeper tissue layers. 3D recordings can be produced from the OCT and IVUS recordings using the electromagnetic position sensors. Alongside this, after a general angiograph has been produced by appropriately utilizing the signals of the position sensors, the catheter's course will be imaged using only the IVUS, OCT, and electromagnetic signals, meaning that x-radiation can be reduced. The system supplies important additional medical information about the arteriosclerotic plaque. The position of the catheter's tip can additionally be better checked with the aid of said information. A further advantage gained from integrating atherectomy and OCT is that in this case a separate rinsing device will not have to be provided for OCT because a rinsing agent is already used for the cutter head.

The sensors of the medical therapy device, which in the exemplary embodiment shown is an x-ray device, are read out partially in time-lagged and clocked fashion. A system clock is first defined in which individual system pulses are generated, with switching-on of the x-radiation and activation of magnetic locating following on from said pulse generating. The x-ray detector will be read out after the x-radiation has been switched off and the IVUS data read out simultaneously. The OCT data will then be read out, that taking place simultaneously with reading out of the ECG and the data relating to respiration. The individual sensors will thus be read out or, as the case may be, the catheter device's components controlled in such a way that mutual interference can be precluded. The time-lagged and clocked manner of reading out shown here is herein to be regarded as exemplifying reading out with interference being avoided.

1.-31. (canceled)

32. A catheter device for performing an atherectomy of a patient, comprising:
an atherectomy catheter; and
a stent premounted on the atherectomy catheter,
wherein the premounted stent and the atherectomy catheter is configured as a single integrated unit.

33. The catheter device as claimed in claim 32, wherein the stent is premounted in a vicinity of a tip of the catheter.

34. The catheter device as claimed in claim 33,
wherein the stent is arranged on an expansible balloon located in the vicinity of the tip of the catheter so that the stent is positioned or secured in a position as a function of the expansion of the balloon, or
wherein the stent is at least partially self-deploying.

35. The catheter device as claimed in claim 32,
wherein a material of the stent comprises metal or bioabsorbable material,
wherein the metal is selected from the group consisting of: a stainless steel, a nitinol, and a memory-metal alloy, and
wherein the bioabsorbable material is selected from the group consisting of: a biological material, a magnesium material, a bio-engineering material, and a plastic.

36. The catheter device as claimed in claim 32,
wherein the stent comprises a coating,
wherein the coating is a nano coating or a active component coating, and
wherein the active component coating is selected from the group consisting of: Sirolimus, Paclitaxel, Everolimus, Rapamycin, and FK 506

37. The catheter device as claimed in claim 32, further comprising a drive device that automatically drives the catheter device at a definable speed.

38. The catheter device as claimed in claim 32, wherein the catheter device is navigated:
mechanically by a pull wire, or
magnetically by a magnetic field generated by a permanent magnet or an electromagnet in the catheter device.

39. The catheter device as claimed in claim 32, further comprising:
a sensor that is selected from the group consisting of: an OCT sensor, an IVUS sensor, a position sensor, and combinations thereof, and
an image processing unit that creates a combined 2D or 3D image based on data from the sensors, wherein the data from the sensors are read out temporally offset to avoid mutual interference.

40. The catheter device as claimed in claim 39, wherein the position sensor is located at a tip of the catheter device and is an electromagnetic sensor comprising:
a transmitting coil in the catheter device and an external receiver coil, or
an external transmitting coil and a receiver coil in the catheter device.

41. The catheter device as claimed in claim 40, wherein the receiver coil comprises an iron core and is a receiving antenna or an electromagnet for a magnetic navigation.

42. The catheter device as claimed in claim 39, wherein the OCT sensor or the IVUS sensor is:
oriented to a side referred to a longitudinal axis of the catheter device, and
separately or jointly rotatable around the longitudinal axis of the catheter device

43. The catheter device as claimed in claim 39, wherein the catheter device and the sensors are electrically decoupled from a power line voltage.

44. The catheter device as claimed in claim 32,
wherein the catheter device comprises a plurality of transmitting coils or a plurality of receiver coils,
wherein the transmitting coils or the receiver coils are arranged orthogonally or at an angle,
wherein the angle is 60°.

45. The catheter device as claimed in claim 32,
wherein the catheter device comprises a coating, and
wherein the coating is a thin-film layer consisting of a silicon material or a conductive nano material.

46. The catheter device as claimed in claim 32,
wherein the image processing unit:
approximates a center line or an envelope curve of a part of a body of the patient being examined, and
registers a 3D image recording of the catheter device with an anatomic image data of the patient according to the center line or the envelope curve,
wherein the anatomic image data is selected from the group consisting of: a 3D angiography data, a computer-assisted tomography data, a nuclear magnetic resonance tomography data, and
wherein the 3D image recording of the catheter device and the anatomic image data is translated into a common coordinate system.

47. The catheter device as claimed in claim 32, wherein a motion artifact caused by breathing or a motion of a moving organ of the patient is determined by registering a frequency or an amplitude of the motion and is computationally corrected.

48. The catheter device as claimed in claim 32, further comprising:
an x-ray marker,
an inflatable balloon arranged at a tip of the catheter device that supports positioning the catheter device, and
a temperature sensor or a pressure sensor arranged at the tip of the catheter device.

49. A medical therapy device for performing an atherectomy of a patient, comprising:
an x-ray imaging device that captures an x-ray image of the patient;
an atherectomy catheter that performs the atherectomy; and
a stent premounted on the atherectomy catheter that supports the vessel after the treatment.

50. A method for monitoring an atherectomy treatment of a patient by an atherectomy catheter, comprising:
integrating an OCT sensor and a stent to the atherectomy catheter;
inserting the integrated catheter into the patient;
capturing an OCT image of the patient by the OCT sensor;
performing the atherectomy treatment by the integrated catheter based on the OCT image; and
placing the stent at a location of the treatment after the treatment.

51. The method as claimed in claim 50, further comprising:
integrating an IVUS sensor or a position sensors to the atherectomy catheter,
connecting the OCT sensor or the IVUS sensor or the position sensor to an image processing unit, and
creating a combined 2D or 3D image recording based on data from the OCT sensor, the IVUS sensor, or the position sensor.
